(19) United States
(12) Patent Application Publication
(10) Pub. No.: US 2021/0030931 A1
(43) Pub. Date: Feb. 4, 2021

(54) DISPOSABLE PULSE LAVAGE/SUCTION DEVICES AND SYSTEMS FOR ORTHOPAEDIC SURGERY

(71) Applicant: HEMODIA, Labège (FR)

(72) Inventor: William SANT, Montgiscard (FR)

(21) Appl. No.: 16/966,709

(22) PCT Filed: Feb. 1, 2019
(86) PCT No.: PCT/FR2019/050236

§ 371 Date: Sep. 11, 2020

(30) Foreign Application Priority Data
Feb. 1, 2018 (FR) 18 50863

(51) Int. Cl. A61M 1/00; A61M 3/02

(52) U.S. Cl. CPC A61M 1/0064; A61M 1/0084; A61M 2250/00; A61M 3/0275; A61M 3/0279; A61M 3/0258

(57) ABSTRACT

The invention relates to disposable pulse lavage/suction devices (100, 400, 600) and systems for orthopaedic surgery. The general principle of the invention starts from the need to provide irrigation and/or suction devices and systems, having a low environmental impact. For this purpose, it is desirable that the disposable elements of such devices and systems can be easily incinerated and/or recycled. To this end, the inventors have identified that the motor, the pump and the battery/cell of said devices and systems should not be part of the disposable elements. The inventors have noted that in hospital operating theatres or similar environments, electromedical apparatuses are found that comprise a pump driven by a motor. Said electromedical devices are powered by the mains. The invention proposes irrigation and/or suction devices and systems that do not directly consume electrical power in order to ensure the operation thereof, but that use the electrical power supply of electromedical apparatuses present in the hospital operating theatres.

### TECHNICAL FIELD

The present invention relates to the field of traumatological or orthopaedic surgery. More specifically, it relates to pulse irrigation and/or suction devices and systems that can be used in hospital operating rooms or similar environments.

### PRIOR ART

High-pressure pulse lavage devices are regularly used during traumatic or orthopaedic interventions for the debridement of bone surfaces as well as for the cleansing of traumatisms and other soft tissue wounds. For example, in the context of an operation for implanting or removing a hip or knee implant, a surgeon is required to use such a system to remove the blood, bone chips and debris appearing during the surgical procedure.

The pulse lavage systems of the prior art, such as that described in patent EP2928516B1, generally comprise a gun-shaped handpiece coupled with an assembly of tubing. It is known that the whole system is single-use and entirely disposable.

In the prior art, the handpiece comprises at least one pump associated with the tubing as well as a motor for driving the pump. Furthermore, the handpiece is powered by a battery that is removably attached to the handpiece, or by a cell that is non-removably attached to the handpiece.

When a battery is removably attached to the handpiece, the battery normally has a significant storage capacity in order to be able to electrically drive the pump sufficiently long enough with a single complete charge of the battery. Yet, such a battery is heavy and fairly cumbersome when same is attached to the handpiece. Furthermore, the removal thereof from the handpiece, then the transport thereof to the recharging site, then the transport thereof in the opposite direction, then the re-installation thereof on the handpiece constitute tedious operations.

When a cell is non-removably attached to the handpiece, the cell is thrown away with the whole lavage system. This is not satisfactory for environmental protection.

Furthermore, in the two preceding scenarios, the drive motor of the pump is also thrown away with the whole lavage system. This is also not satisfactory for environmental protection.

### SUMMARY OF THE INVENTION

The present invention therefore aims to overcome the aforementioned drawbacks by procuring, in a first aspect of the invention, a disposable pulse lavage device intended to be used with an electromedical apparatus for orthopaedic surgery. The electromedical apparatus comprises a peristaltic pump and a control motor to drive the peristaltic pump. The peristaltic pump comprises a pinching member provided to apply a pressure on a tube. The lavage device comprises:
a handpiece,
an irrigation tube configured to extend from the handpiece to an irrigation fluid source, and
an irrigation tip mounted at a front end of the handpiece, the irrigation tip comprising an irrigation port connected to the irrigation tube through a passage formed within the handpiece.

In the first aspect of the invention, the irrigation tube is configured to be placed in contact with the pinching member so that the driving of the peristaltic pump causes a pressure to be applied by the pinching member on the irrigation tube to make the irrigation fluid flow from the irrigation fluid source to the irrigation port.

In a second aspect of the invention, it is proposed a disposable pulse suction device intended to be used with an electromedical apparatus for orthopaedic surgery similar to same used in the first aspect. The lavage device comprises:
a handpiece,
a suction tube extending from the handpiece to a recovery circuit, and
a suction tip mounted at a front end of the handpiece, the suction tip comprising a suction port connected to the suction tube through a passage formed within the handpiece, the suction port being provided to come into contact with an area to be treated of the human or animal organism comprising matter to be sucked out.

In the second aspect of the invention, the suction tube is configured to be placed in contact with the pinching member so that the driving of the peristaltic pump, causes a pressure to be applied by the pinching member on the suction tube to make matter to be sucked out flow from the suction port to the recovery circuit.

In a third aspect of the invention, it is proposed a disposable pulse lavage/suction device combining the first and second aspects of the invention.

Finally, in a fourth aspect of the invention, it is proposed a pulse irrigation and/or suction system comprising one or more devices according to the first, second and/or third aspects of the invention. Furthermore, in the pulse irrigation and/or suction system comprises at least one control pedal provided to be coupled with a motor of the electromedical apparatus. In the fourth aspect of the invention, the control pedal is configured to control the force for driving the motor of the electromedical apparatus.

### BRIEF DESCRIPTION OF THE DRAWINGS

Other features and advantages of the invention will be better understood upon reading the following description and with reference to the appended drawings, given by way of illustrative and non-limiting examples.

FIG. 1 shows a disposable pulse lavage device according to the invention.

FIG. 2 shows an example of handpiece according to the invention.

FIG. 3 shows a disposable pulse suction device according to the invention.

FIG. 4 shows a disposable pulse lavage/suction device according to the invention.

For reasons of clarity, the elements shown are not to scale, in relation to one another, unless otherwise specified.

### DESCRIPTION OF EMBODIMENTS

The general principle of the invention starts from the need to provide irrigation and/or suction devices and systems, having a low environmental impact. For this purpose, it is desirable that the disposable elements of such devices and systems can be easily incinerated and/or recycled. To this end, the inventors have identified that the motor, the pump and the battery/cell of said devices and systems should not be part of the disposable elements. The inventors have noted that in hospital operating rooms or similar environments, electromedical apparatuses are found that comprise a pump driven by a motor. Said electromedical devices are powered by the mains. The invention proposes irrigation and/or suction devices and systems that do not directly consume electrical power in order to ensure the operation thereof, but that use the electrical power supply of electromedical apparatuses present in the hospital operating rooms.

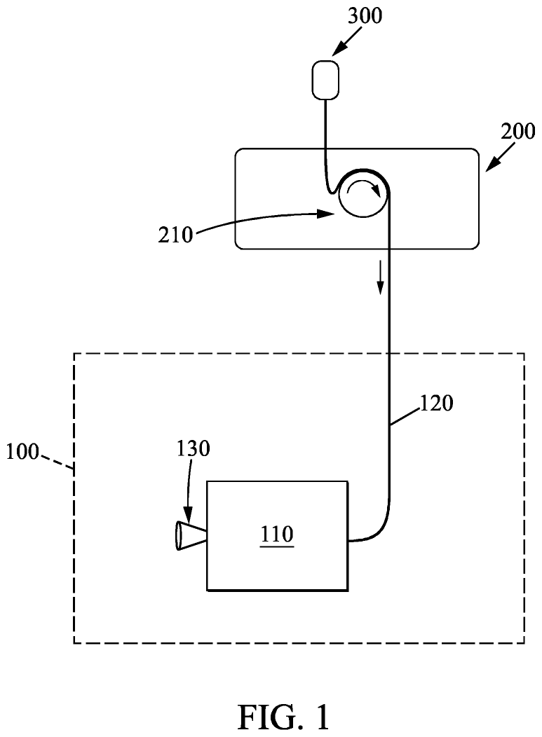

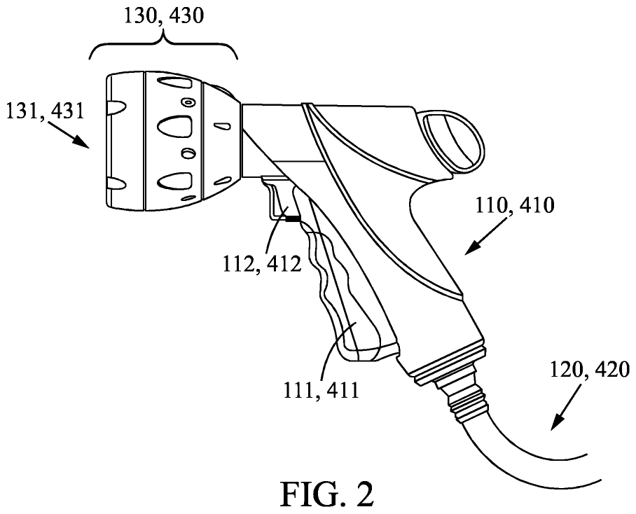

FIG. 1 in relation with FIG. 2 illustrates a first implementation of the invention.

The example in FIG. 1 shows a disposable pulse lavage device 100. The lavage device 100 is intended to be used with an electromedical apparatus for orthopaedic surgery. For example, the electromedical apparatus for orthopaedic surgery is an irrigation pump for arthroscopy 200. The irrigation pump for arthroscopy 200 in FIG. 1 comprises a peristaltic pump 210 and a control motor (not shown) for driving the peristaltic pump 210.

The lavage device 100 comprises a handpiece 110, an irrigation tube 120 and an irrigation tip 130.

In one particular implementation, in relation with FIG. 2, the handpiece 110 has a gun shape.

Returning to FIG. 1, the irrigation tube 120 extends from the handpiece 110 to an irrigation fluid source 300.

The irrigation tip 130 is mounted at a front end of the handpiece 110. Furthermore, the irrigation tip 130 comprises an irrigation port 131 connected to the irrigation tube 120 through a passage (not shown) formed within the handpiece 110. In one particular implementation, the irrigation tip 130 is removably coupled with a rotary nozzle with rotor or with a variable jet nozzle for varying the pressure of a fluid passing through the irrigation port 131.

In FIG. 2, the handpiece 110 further comprises a trigger 111 moveable between an actuated position wherein the passage formed within the handpiece 110 is open and an unactuated position wherein the passage formed within the handpiece 110 is closed. In this way, it is possible to control the ejection of a fluid passing through the passage formed within the handpiece 110.

In one particular implementation, the handpiece 110 also comprises a locking element 112 moveable between a locked position wherein the locking element 112 prevents the movement of the trigger 111, and an unlocked position. In this way, it is possible to block the trigger 111 in a preferred position.

In the first implementation of the invention in FIG. 1, the irrigation tube 120 is configured to be coupled with a pinching member (not shown) of the peristaltic pump 210 so that when the peristaltic pump 210 is driven, the irrigation fluid is made to flow from the irrigation fluid source 300 towards the irrigation port 131. In one example, the pinching member is a presser finger or a roller of the peristaltic pump 210 that is configured to squeeze the irrigation tube 120 in response to a squeeze command of the electromedical apparatus for orthopaedic surgery 200. It is considered here that the squeeze command of the surgery electromedical apparatus makes it possible to define an ejection flow rate of the irrigation fluid flowing in the irrigation tube 120. In one particular implementation, stretching means tension the irrigation tube 120 before the pinching member squeezes same.

The first implementation of the invention enables the pulse lavage device 100 to only comprise disposable elements that can be easily incinerated and/or recycled. Furthermore, said implementation has positive effects in terms of environmental protection. Indeed, the pulse lavage device 100 does not comprise a motor, because same is included in the electromedical apparatus for orthopaedic surgery 200, just like the peristaltic pump 210. Thus, neither the motor nor the peristaltic pump 210 will be thrown away after pulse lavage device 100 use. Furthermore, the pulse lavage device 100 does not directly require electrical power in order to ensure the lavage function thereof, because it is the electromedical apparatus for orthopaedic surgery 200 that electrically powers the motor driving the peristaltic pump 210. Thus, no battery/cell will be thrown away after pulse lavage device 100 use.

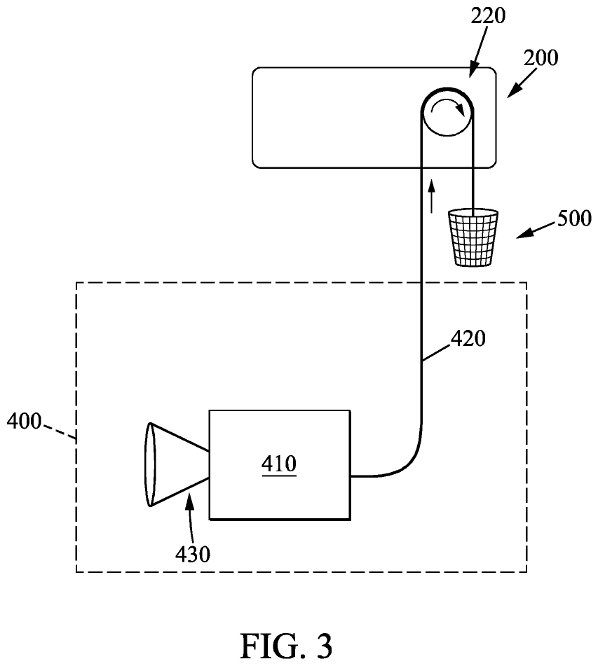

FIG. 3 in relation with FIG. 2 illustrates a second implementation of the invention.

The example in FIG. 3 shows a disposable pulse suction device 400. The disposable pulse suction device 400 is intended to be used with an electromedical apparatus for orthopaedic surgery. For example, the electromedical apparatus for orthopaedic surgery is the irrigation pump for arthroscopy 200 such as described above. The irrigation pump for arthroscopy 200 in FIG. 3 comprises a peristaltic pump 220 and a control motor (not shown) for driving the peristaltic pump 220. The peristaltic pump 220 is similar to the peristaltic pump 210 such as described above.

The disposable pulse suction device 400 comprises a handpiece 410, a suction tube 420 and a suction tip 430.

In one particular implementation, in relation with FIG. 2, the handpiece 410 has a gun shape.

Returning to FIG. 3, the suction tube 420 extends from the handpiece 410 to a recovery circuit 500.

The suction tip 430 is mounted at a front end of the handpiece 410. Furthermore, the suction tip 430 comprises a suction port 431 connected to the suction tube 420 through a passage (not shown) formed within the handpiece 410. The suction port 431 is provided to come into contact with an area to be treated of the human or animal organism comprising matter to be sucked out. In one particular implementation, the suction tip 430 is removably coupled with a suction nozzle for maximising the quantity of matter to be sucked out.

In FIG. 2, the handpiece 410 further comprises a trigger 411 moveable between an actuated position wherein the passage formed within the handpiece 410 is open and an unactuated position wherein the passage formed within the handpiece 410 is closed. In this way, it is possible to control the suction of matter passing through the passage formed within the handpiece 110.

In one particular implementation, the handpiece 410 also comprises a locking element 412 moveable between a locked position wherein the locking element 412 prevents the movement of the trigger 411, and an unlocked position. In this way, it is possible to block the trigger 411 in a preferred position.

In the second implementation of the invention in FIG. 3, the suction tube 420 is configured to be coupled with a pinching member (not shown) of the peristaltic pump 220 so that when the peristaltic pump 220 is driven, matter to be sucked out is made to flow from the suction port 431 towards the recovery circuit 500. In one example, the pinching member is a presser finger or a roller of the peristaltic pump 220 that is configured to squeeze the suction tube 420 in response to a squeeze command of the electromedical apparatus for orthopaedic surgery 200. It is considered here that the squeeze command of the surgery electromedical apparatus makes it possible to define a suction flow rate of the matter flowing in the suction tube 420. In one particular implementation, stretching means tension the irrigation tube 120 before the pinching member squeezes same.

The second implementation of the invention enables the pulse suction device 400 to only comprise disposable elements that can be easily incinerated and/or recycled. Furthermore, said implementation has the same effects in terms of environmental protection mentioned above.

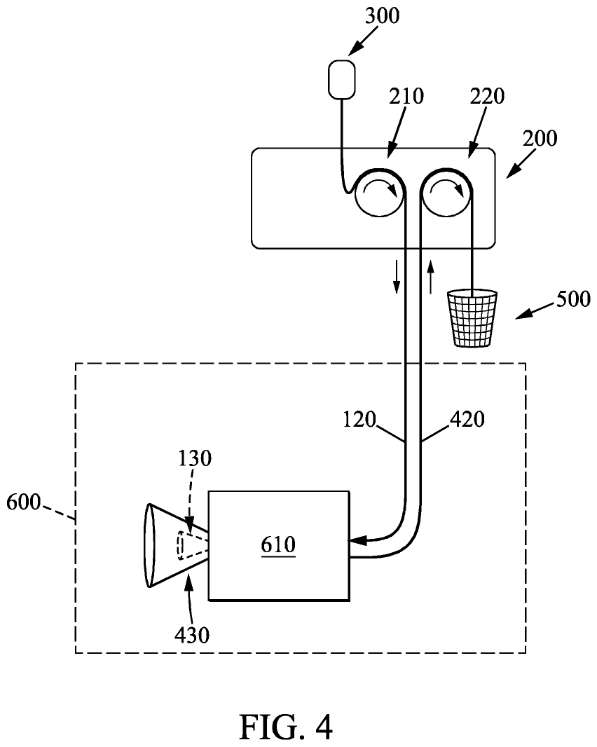

FIG. 4 illustrates a third implementation of the invention that combines the first and the second implementation of the invention. The example in FIG. 4 thus shows a disposable pulse lavage/suction device 600 for which the elements already described above will not be described again.

The disposable pulse lavage/suction device 600 comprises a single handpiece 610 similar to the handpieces 110 and 410, from which extend the irrigation tube 120 and the suction tube 420.

In one particular implementation, the irrigation tip 130 is disposed in the suction tip 430.

It is also envisaged a pulse irrigation and/or suction system for orthopaedic surgery comprising at least one disposable device according to the first, second and third implementations, and at least one control pedal provided to be coupled with a motor of the electromedical apparatus.

In the system according to the invention, the control pedal is configured to control the force for driving the motor of the electromedical apparatus. In this way, it is possible to control the driving of the peristaltic pump 210, 220 so as to accelerate or slow down the flow rate of the fluids or matter flowing in the irrigation tubes 120 or suction tubes 420.

In one particular implementation, the control pedal comprises two channels or four channels. For example, when the control pedal comprises two channels, one of the pedals may be configured to increase the flow rate by 50% whereas the other is configured to reduce the flow rate by 25%. Of course, other percentage configurations are possible.

The present invention has been described and illustrated in the present detailed description and in the figures. However, the present invention is not limited to the embodiments presented. Thus, other alternatives and embodiments may be deduced and implemented by the person skilled in the art upon reading the present description and appended figures.

1-10. (canceled)

11. Disposable pulse lavage device intended to be used with an electromedical apparatus for orthopaedic surgery, the electromedical apparatus comprising a peristaltic pump and a control motor to drive the peristaltic pump, the peristaltic pump comprising a pinching member provided to apply a pressure on a tube, the lavage device comprising:
a handpiece,
an irrigation tube configured to extend from the handpiece to an irrigation fluid source, and
an irrigation tip mounted at a front end of the handpiece, the irrigation tip comprising an irrigation port connected to the irrigation tube through a passage formed within the handpiece,
wherein the irrigation tube is configured to be placed in contact with the pinching member so that the driving of the peristaltic pump causes a pressure to be applied by the pinching member on the irrigation tube to make the irrigation fluid flow from the irrigation fluid source towards the irrigation port.

12. The device according to claim 11, wherein the handpiece further comprises a trigger moveable between an actuated position wherein the passage formed within the handpiece is open and an unactuated position wherein the passage formed within the handpiece is closed.

13. The device according to claim 11, wherein the handpiece has a gun shape.

14. The device according to claim 11, wherein the electromedical apparatus is an arthroscopy pump.

15. Disposable pulse suction device intended to be used with an electromedical apparatus for orthopaedic surgery, the electromedical apparatus comprising a peristaltic pump and a control motor to drive the peristaltic pump, the peristaltic pump comprising a pinching member provided to apply a pressure on a tube, the suction device comprising:
a handpiece,
a suction tube extending from the handpiece to a recovery circuit, and
a suction tip mounted at a front end of the handpiece, the suction tip comprising a suction port connected to the suction tube through a passage formed within the handpiece, the suction port being provided to come into contact with an area to be treated of the human or animal organism comprising matter to be sucked out,
wherein the suction tube is configured to be placed in contact with the pinching member so that the driving of the peristaltic pump, causes a pressure to be applied by the pinching member on the suction tube to make matter to be sucked out flow from the suction port towards the recovery circuit.

16. The device according to claim 15, wherein the handpiece further comprises a trigger moveable between an actuated position wherein the passage formed within the handpiece is open and an unactuated position wherein the passage formed within the handpiece is closed.

17. The device according to claim 15, wherein the handpiece has a gun shape.

18. The device according to claim 15, wherein the electromedical apparatus is an arthroscopy pump.

19. Disposable pulse lavage/suction device intended to be used with an electromedical apparatus for orthopaedic surgery, the electromedical apparatus comprising a first and a second peristaltic pump and a first and a second respective control motor to drive the first and second peristaltic pumps, each of the first and second peristaltic pumps comprising a pinching member provided to apply a pressure on a tube, the lavage/suction device comprising:
a handpiece,
an irrigation tube extending from the handpiece to an irrigation fluid source,
an irrigation tip mounted at a front end of the handpiece, the irrigation tip comprising an irrigation port connected to the irrigation tube through a passage formed within the handpiece,
a suction tube configured to extend from the handpiece to a recovery circuit, and
a suction tip mounted at a front end of the handpiece, the suction tip comprising a suction port connected to the suction tube through a passage formed within the handpiece, the suction port being provided to come into contact with an area to be treated comprising matter to be sucked out,
wherein,
the irrigation tube is configured to be placed in contact with the pinching member so that the driving of the peristaltic pump causes a pressure to be applied by the pinching member on the irrigation tube to make the irrigation fluid flow from the irrigation fluid source towards the irrigation port, and
the suction tube is configured to be placed in contact with the pinching member so that the driving of the peristaltic pump causes a pressure to be applied by the pinching member on the suction tube to make matter to be sucked out flow from the suction port towards the recovery circuit.

20. The device according to claim 19, wherein the irrigation tip is disposed in the suction tip.

21. The device according to claim 19, wherein the handpiece further comprises a trigger moveable between an actuated position wherein the passage formed within the handpiece is open and an unactuated position wherein the passage formed within the handpiece is closed.

22. The device according to claim 21, further comprising:
a locking element moveable between a locked position wherein the locking element prevents the movement of the trigger, and an unlocked position.

23. The device according to claim 19, wherein the handpiece has a gun shape.

24. The device according to claim 19, wherein the electromedical apparatus is an arthroscopy pump.

25. Irrigation and/or suction system for orthopaedic surgery comprising:
at least one disposable pulse lavage device intended to be used with an electromedical apparatus for orthopaedic surgery, the electromedical apparatus comprising a peristaltic pump and a control motor to drive the peristaltic pump, the peristaltic pump comprising a pinching member provided to apply a pressure on a tube, the lavage device comprising:
a handpiece,
one of an irrigation tube configured to extend from the handpiece to an irrigation fluid source or a suction tube configured to extend from the handpiece to a recovery circuit, and
one of an irrigation tip and a suction tip, mounted at a front end of the handpiece, the irrigation tip or suction tip comprising respectively an irrigation port or suction port connected to respectively the irrigation tube or suction tube through a passage formed within the handpiece, wherein the irrigation tube or suction tube is configured to be placed in contact with the pinching member so that the driving of the peristaltic pump causes a pressure to be applied by the pinching member on the irrigation tube or suction tube, to make the irrigation fluid flow from the irrigation fluid source towards the irrigation port or to make matter to be sucked out flow from the suction port towards the recovery circuit, respectively, and
at least one control pedal provided to be coupled with a motor of the electromedical apparatus, and configured to control the driving force of the motor of the electromedical apparatus.

26. The system according to claim 25, wherein the control pedal comprises two channels or four channels.
